(19) United States
(12) Patent Application Publication
(10) Pub. No.: US 2007/0033835 A1
(43) Pub. Date: Feb. 15, 2007

(54) Insole arrangement; footwear with insole arrangement; and, method of preparation

(76) Inventor: Walter Thomas Bray JR., Reynoldsburg, OH (US)

Correspondence Address: MERCHANT & GOULD PC, P.O. BOX 2903, MINNEAPOLIS, MN, 55402-0903, US

(21) Appl. No.: 11/498,972

(22) Filed: Aug. 2, 2006

Related U.S. Application Data
(60) Provisional application No. 60/705,117, filed on Aug. 2, 2005.

(51) Int. Cl. A43B 13/18; A43B 13/38; A61F 5/14

(52) U.S. Cl. 36/44; 36/153; 36/28

(57) ABSTRACT

A footwear member with a desired insole arrangement is described. The footwear arrangement comprises a footwear shell including an insole insert arrangement, in which the insole insert arrangement includes: an insole base structure with a heel cut therein; and, a heel cushion arrangement positioned in the insole arrangement with a portion projecting through the heel cut. A typical heel cushion arrangement would include a gel pack with an exposed upper surface. Methods of assembly are described.

### CROSS REFERENCE TO RELATED APPLICATIONS

The present application claims priority to U.S. Provisional Patent Application Serial No. 60/705,117, filed on Aug. 2, 2005, the disclosure of which is hereby incorporated by reference in its entirety.

### FIELD OF THE DISCLOSURE

The present disclosure relates to footwear. It particularly concerns footwear insole constructions and footwear arrangements which use the footwear insole constructions. Methods of manufacture and examples are also described. The techniques described are particularly useful in providing slippers.

### BACKGROUND

The present disclosure relates to footwear. The techniques described will typically be applied in a type of footwear generally referred to as “slippers”. A typical characterization of slippers is that they are non-sock style footwear with a sole and an upper typically not constructed to survive the rigors to which everyday walking shoes are applied. They are usually softer than shoes and constructed for comfort, and for ease to put on. Many are machine washable in ordinary clothes washers. A typical characteristic of slippers is that they are not manufactured with a last, which is often a necessary device when manufacturing a shoe having a hard sole and a very robust, for example leather, upper. Slippers typically are slip-on, without laces or straps.

Typical slippers have lightweight polymeric soles with fabric uppers, although alternatives are possible.

In general, the slipper footwear industry is a competitive, crowded, industry with significant competition relating to providing alternate slipper choices for the consumer with respect to: comfort; aesthetic appeal; cost; ease of manufacture; and, related factors.

In U.S. Pat. No. 6,931,763 B2, filed Aug. 5, 2003, issued Aug. 23, 2005, and incorporated in its entirety herein by reference, unique slipper arrangements and methods for manufacture are described. The slipper arrangements of U.S. Pat. No. 6,931,763, generally include a sole, an upper, and an insole insert, in which the insole insert is a result of compression molding a structure including a foam layer to provide a desirable contour. Useable techniques and materials to accomplish desired results, are described in that reference.

Continual improvements are ongoing with respect to: providing alternative choices for the consumer, and, improving manufacture.

### SUMMARY

According to the present disclosure a footwear member (or piece) is provided. The footwear member (or piece) is a member of a pair of footwear, such as a pair of slippers. The techniques described herein are particularly adapted for use in forming slip-on footwear, and would typically not be utilized for shoes that would have a hard sole, a leather upper and laces or straps.

In general, the footwear member comprises a footwear shell and a footwear insole insert structure arrangement (or footwear sole insert structure). The footwear shell typically includes at least a sole and an upper. The sole and upper together define a footwear shell interior. The upper can be configured with a closed toe or an open toe construction.

The footwear insole insert structure arrangement is secured within the footwear shell interior, for example through use of an adhesive, stitching or both. The footwear insole insert structure arrangement can be nonremovably secured within the footwear shell interior, so that the footwear insole insert structure arrangement cannot removed without damaging either the insole insert structure arrangement or the footwear shell to which it is secured. The footwear insole insert structure arrangement generally includes an insole base structure and a heel center cushion arrangement.

The insole base structure includes a heel cut therethrough. Although alternatives are possible, the heel cut is typically an aperture in a heel region of the insole base structure, and is surrounded by other portions of the insole base structure. However alternate heel cut arrangements are possible.

The heel center cushion arrangement is positioned within the heel cut, defining an exposed upper surface of the heel center cushion arrangement for contact with the heel of a wearer. By “heel of a wearer” in this context, reference is meant to a heel portion of a wearer's foot whether the foot is covered by a sock, etc., or otherwise. By the term “exposed upper surface” in this manner, it is meant that the upper surface of the heel center cushion arrangement is not covered by a portion of the insole base structure, or any other sole insert placed within the shell interior; the “upper surface,” being a surface that projects upwardly into the footwear shell.

Although alternatives are possible, typically the insole base structure includes a heel periphery support portion, a central arch region and a front section. The heel periphery support portion is a portion of the base structure positioned under a peripheral portion of the wearer's heel. In this context the word “peripheral portion” refers to a portion of the wearer's heel that does not overlap the heel center cushion arrangement. In a typical footwear member incorporating techniques according to the present disclosure, the heel center cushion arrangement defines an exposed upper surface which is smaller than the area of a wearer's heel; and, peripheral heel support is also provided by the insole base structure in regions where cushioning is not provided by the heel center cushion arrangement.

The central arch region of the insole base structure is centrally positioned under the arch of a wearer's foot, in use. The front section is a portion typically positioned under the ball of a wearer's foot and the toes of a wearer's foot.

The insole base structure can include a rim portion. The rim portion would typically project upwardly from an upper surface of the insole base structure, at least around regions of the central arch region and the heel periphery support region. The rim can be configured to project upwardly completely around the upper surface of the insole base structure, if desired.

The heel periphery support portion can be a contoured portion, with raised regions, for example spaced raised cushion regions separated by regions of reduced height and/or cushion. These raised regions can be provided with a desirable cushion characteristic, for support to the periphery of a wearer's heel.

The exposed upper surface of the heel center cushion arrangement may be positioned above the upper surface of the raised regions within the heel periphery support portion. For example, the exposed upper surface of the heel center cushion arrangement may be 1 to 3 mm higher than the upper surface of the raised regions. Alternatively, the raised regions and the exposed upper surface of the heel center cushion arrangement may be at the same height. In another alternative, the upper surface of the raised regions is higher than the exposed upper surface of the heel center cushion arrangement.

The insole base structure would typically comprise a compression molded laminate of fabric and foam, although alternatives are possible.

The heel center cushion arrangement would typically comprise a gel pack. The gel pack would typically a comprise a package or envelope, in which a portion of a polymeric cushion material having desirable cushion properties would be included. An example of a polymeric cushion material described herein is a polyurethane cushion.

In an example shown, the heel center cushion arrangement comprises a gel pack positioned with a cover piece, in particular a foam piece, underneath. The cover piece would typically be secured to an under side of the insole base structure.

In an example shown, the heel cut is an aperture which includes a recessed lip at a location adjacent the under (bottom) surface of the insole base structure. The recessed lip is used for mechanical engagement (through overlap) with an outwardly projecting annular lip on the gel pack, to help secure the gel pack in position.

Although alternatives are possible, the exposed upper perimeter area of the heel center cushion arrangement (and also the perimeter area defined by heel cut) in many applications would be an area of at least 1100 square millimeters (sq. mm.), and not more than 1800 sq. mm. The term “perimeter area” and variants thereof in this context, is meant to refer to an area defined by a perimeter of the exposed upper surface of the heel center cushion arrangement (or the area defined by the heel cut). Often the area would be within the range of 1200-1600 sq. mm., inclusive; a typical example being within the range of 1250-1500 sq. mm., inclusive.

Although the heel center cushion arrangement defining the exposed upper surface can be provided in a variety of shapes, a typical shape would be an oval shape, an example being an elliptical shape.

### BRIEF DESCRIPTIONS OF THE DRAWINGS

FIG. 1 is a schematic perspective view of a slipper shell construction according to the principles of the present disclosure, the shell construction of FIG. 1 being ready to receive an insole insert structure arrangement according to the present disclosure therein.

FIG. 2 is a schematic perspective exploded view of materials usable to form a portion of an insole insert structure arrangement according to the present disclosure.

FIG. 3 is a schematic perspective view of an insole base structure of an insole structure arrangement formed from the construction of FIG. 2; the insole base structure of FIG. 3 being devoid of a heel center cushion arrangement.

FIG. 4 is a schematic bottom plan view of the insole base structure of FIG. 3.

FIG. 4A is a schematic, fragmentary cross-sectional view of a portion of FIG. 4 and shown inverted relative to FIG. 4 (and thus upright); FIG. 4A being taken along line 4A-4A, FIG. 4.

FIG. 5 is an exploded schematic view of a heel center cushion arrangement usable to form an insole insert structure arrangement according to the present disclosure.

FIG. 6. is a view of the structure of FIG. 5, shown in a stacked form and in cross-section.

FIG. 7 is a schematic view of an assembled insole insert structure arrangement comprising the sole base structure of FIG. 3 having the heel cushion arrangement of FIG. 6 positioned therein.

FIG. 8 is a perspective schematic view of an assembled footwear member comprising the footwear shell of FIG. 1 having the insole insert structure arrangement of FIG. 7 positioned therein.

### DETAILED DESCRIPTION OF THE PREFERRED EMBODIMENT

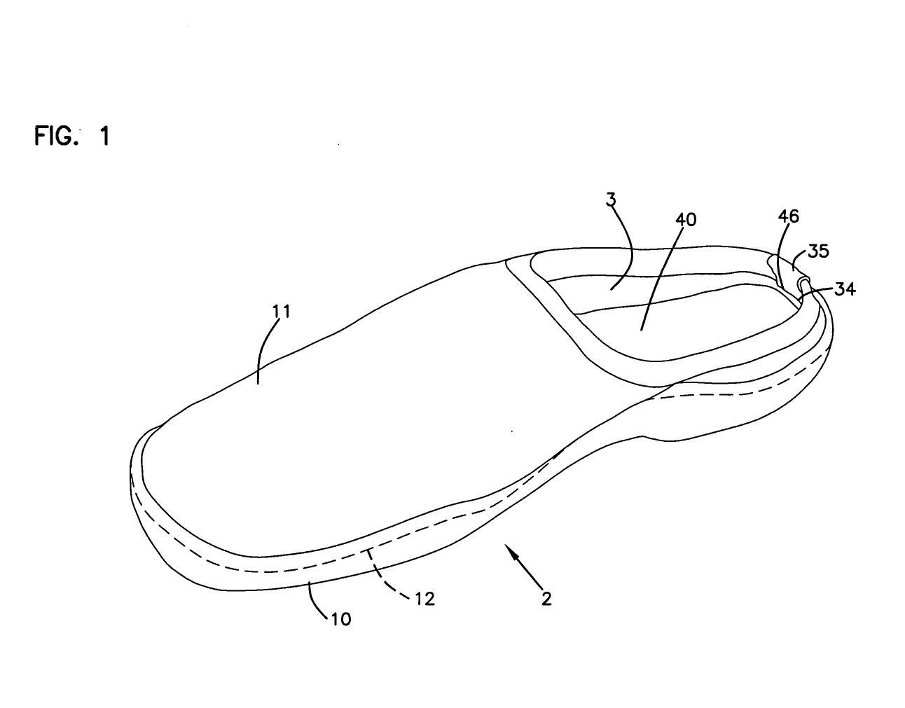

As indicated above, the present disclosure relates to: a footwear insole insert arrangement or construction for use in footwear; resulting footwear assemblies; and, methods of preparation of such arrangements. The techniques can be used to form a variety of types of footwear, but were particularly adapted for use in forming slippers. In FIG. 8, discussed below, an assembled footwear member or piece, in particular a slipper, made according to the present disclosure is provided. In FIGS. 1-7, subparts usable to provide the assembled footwear member of FIG. 8, are shown. From the later descriptions of FIGS. 1-7, it will be apparent how structure according to FIG. 8 can be assembled.

### I. An Example Footwear Assembly; FIG. 8

The reference numeral 1, FIG. 8, generally designates a footwear member according to the present disclosure. The term “footwear member” as used herein, is generally meant to refer to a member of a footwear pair. The term “footwear piece” is sometimes alternatively used to provide this designation.

Referring to FIG. 8, footwear member 1 generally comprises a footwear shell 2 (defining interior 3) having a footwear sole insert structure arrangement 5, according to the present disclosure, positioned therein.

Referring to FIG. 8, the footwear shell 2 generally comprises a sole (or outsole) 10 and an upper 11. The upper 11 will typically comprise a fabric construction, although alternatives are possible. The upper 11 can be formed to have either an open toed or closed toed configuration, with the principles of the present disclosure. The sole 10 will typically comprise a flexible polymeric sole member and may include fabric coverings or fabric components therein. Alternatives are possible.

In typical constructions, the upper 11 is secured to the sole 10 by stitching 12 although in some assemblies adhesives can be used.

Conventional, known, methods, materials and arrangements can be used for the sole 10 and upper 11; however materials, methods and arrangements yet to be developed can also be used. The selection of materials for the sole 10 and upper 11 is generally a matter of choice for factors not directly related to inserts according to this disclosure and a wide variety can be utilized with techniques according to the present disclosure.

The sole 10 can include an upper, cushion, sole piece which, in the assembled footwear member 8, will be positioned under the footwear sole insert structure arrangement 5, to provide additional padding or cushioning, if desired.

Referring still to FIG. 8, the footwear sole structure arrangement 5 is generally permanently secured within an interior 3, for example using an adhesive, although in some instances stitching could be used. The footwear sole insert structure arrangement 5 typically includes: heel center portion 15; heel periphery support portion 16; center arch region 19 and forward or front section 20; the forward or front section 20 being a portion under a ball and toes of a wearer's foot; in use. Although not required, a typical footwear sole insert structure arrangement 5 will include a rim portion 22 peripherally surrounding, and projecting upwardly from, at least a portion of an adjacent remainder of footwear sole insert structure arrangement 5; typically at least the central or arch region 19 and the heel periphery support portion 16, although variations are possible.

Still referring to FIG. 8, at 25 optional breathe apertures or perforations are shown provided through footwear sole insert structure arrangement 5. Typically such optional breathe perforations or apertures 25 are located in one or more of: the center arch region 19, the forward or front section 20 and a portion of the rim portion 22 adjacent the center arch region 19, although alternatives are possible. Typically such breathe apertures are circular and have a diameter on the order of 0.5-1.5 mm, although alternatives are possible.

For the example shown, the optional breathe apertures are provided in central arch region 19, and a portion of the rim portion 22 adjacent the central arch region 19. Such breathe perforations are used to provide for improved air circulation in the interior of the footwear piece 1, to facilitate comfort. In addition, such perforations 25 help provide flexibility to the central arch region 19.

As will be apparent from the following descriptions, the present disclosure is particularly concerned with the heel center cushion portion 15, and methods for assembling the footwear member 1 with such a heel center cushion portion 15.

### II. Assembly of the Footwear Piece 1, FIGS. 1-7

A. The Footwear Shell; FIG. 1.

Attention is now directed to FIG. 1, in which footwear shell 2 is depicted. In FIG. 1, footwear shell 2 is shown without a footwear sole insert structure arrangement 5 therein. Thus, shell 2 comprises sole 10, and, upper 11 secured to one another, for example by stitching as shown at 12. Again, as mentioned above, shell 2 and thus the resulting footwear piece 1 (FIG. 8) can be constructed by securing the upper 11 to the sole 10 by alternate means, for example with adhesive.

The shell 2 can include a variety of optional features, as chosen by the manufacturer, in accord with the principles of the present disclosure. For example, the shell 2 depicted includes a heel rim 34 with a wrap 35. An alternate or additional loop structure can also be provided at the heel location, for ease of picking up the slippers or sliding them on. Decorative materials can be attached to the upper 11, also.

Also as previously discussed, the upper 11 can be configured as an open toed or closed toed configuration. A typical upper 11 will be devoid of tie laces or mounting straps, as is typical for slippers.

Further, the sole 10 can include an upper sole cushion arrangement at 40, for example comprising an extension of fabric (or foam under fabric) providing a desirable substructure underneath the footwear sole insert structure arrangement 5, FIG. 8, if desired. Such an option sole cushion 40 can be positioned and secured in place, for example, by stitching and/or adhesive.

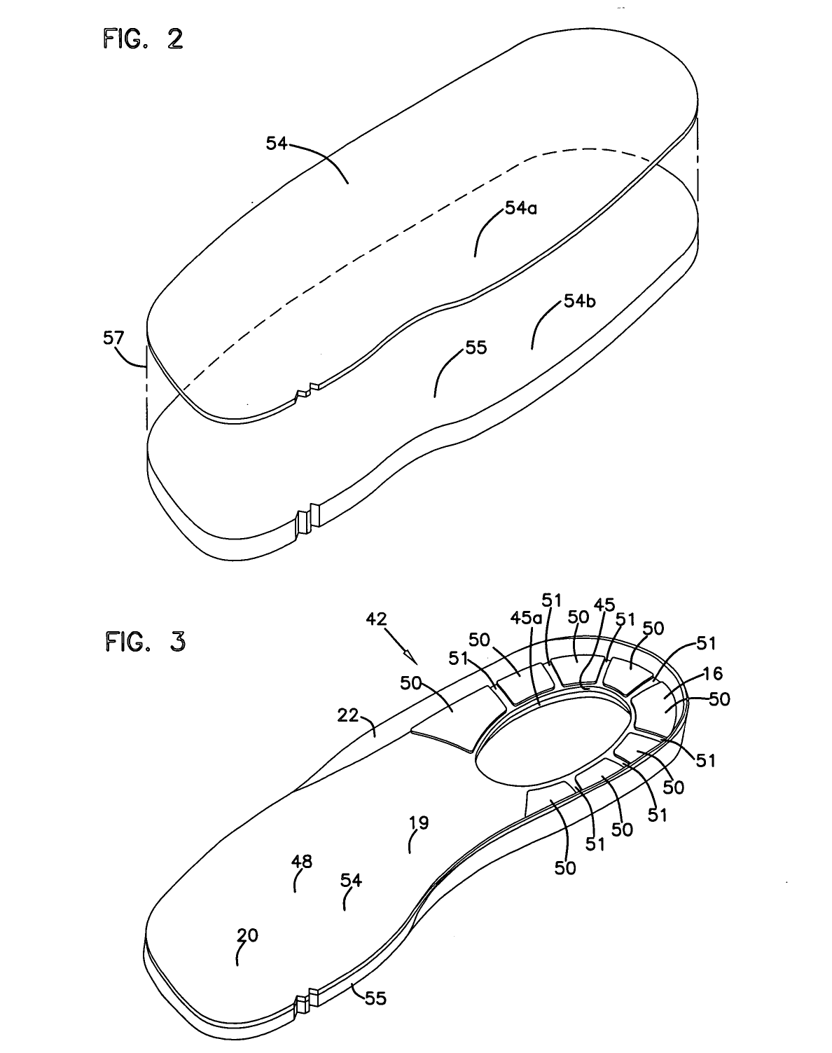

B. Construction of the Footwear Sole Insert Structure Arrangement 5, FIGS. 2-7.

In FIGS. 2-7, preparation of an example footwear sole insert structure arrangement 5 is shown. In FIG. 8, positioning of the sole insert structure within the shell 2, to form the footwear piece 1, is shown and described.

Referring to FIG. 8, in typical applications of the present disclosure, the footwear sole insert structure arrangement will include two general subparts: (a) a heel cushion arrangement 15a, which, when installed, will provide the heel center cushion arrangement or portion 15; and (b) a insole base structure 42 which will form a remainder of the insole insert structure arrangement 5. That is, the typical insole base structure 42 would include: the heel periphery support portion 16; the central arch region 19; the front section 20; and, the rim portion 22, although not all of the features are required.

1. Preparation of Insole Base Structure 42, FIGS. 2-4.

In FIG. 3, a top perspective view of the insole base structure 42 is depicted, in which one can see that the insole base structure 42 includes heel cut 45 therethrough, in a heel portion, for installation of the heel center cushion subassembly therein, at a location which will be under a wearer's heel.

The heel cut 45 may be positioned as an aperture 45a extending through the insole base structure 42 such that a portion of the insole base structure 42 completely surrounds the heel cut 45, thus forming, as the heel cut 45, a heel cut aperture 45a. However, an aperture only surrounded by portions of insole base structure 42 is not required in all instances, and in some instances the heel cut 45 can define a region which is bounded, for example at the rear, not by a portion of insole base structure 42 but rather by the heel portion 46 of the footwear shell 2.

A peripheral perimeter defined by the heel cut 45 (either alone or in combination with a portion of the heel side wall) will typically be at least 1100 sq. mm. and typically not more than 1800 sq. mm., although in some instances alternatives are possible. Usually the perimeter area will be at least 1200 sq. mm., and not more than 1600 sq. mm., inclusive. In typical examples the perimeter area is within the range of 1250-1500 sq. mm., inclusive.

The shape defined by the heel cut 45 is a matter of choice. It is expected that typically an oval shape (a specific example of which is an elliptical shape) will be used, having a long axis and a short axis; the aperture 45a being oriented so that: a long axis of the oval extends along a length of the footwear member 1, FIG. 8, and short axis extends generally perpendicularly thereto. Herein the term “oval” is meant to refer to a shape which has two, narrowly curved, opposite ends and two opposite sides, whether the sides are straight or also curved, whether the ends are of the same curvature or not. The term “elliptical” is meant to refer to a particular oval shape, i.e., a shape corresponding to an ellipse.

Except for the presence of heel cut 45, the insole base structure 42 may, if desired, be constructed generally in accord with the principles described in U.S. Pat. No. 6,931,763 B2, filed Aug. 5, 2003, issued Aug. 23, 2005, and previously incorporated herein by reference. However, it is noted that alternate constructions for the insole base structure can be utilized, in constructing a footwear piece 1 in accord with the present disclosure.

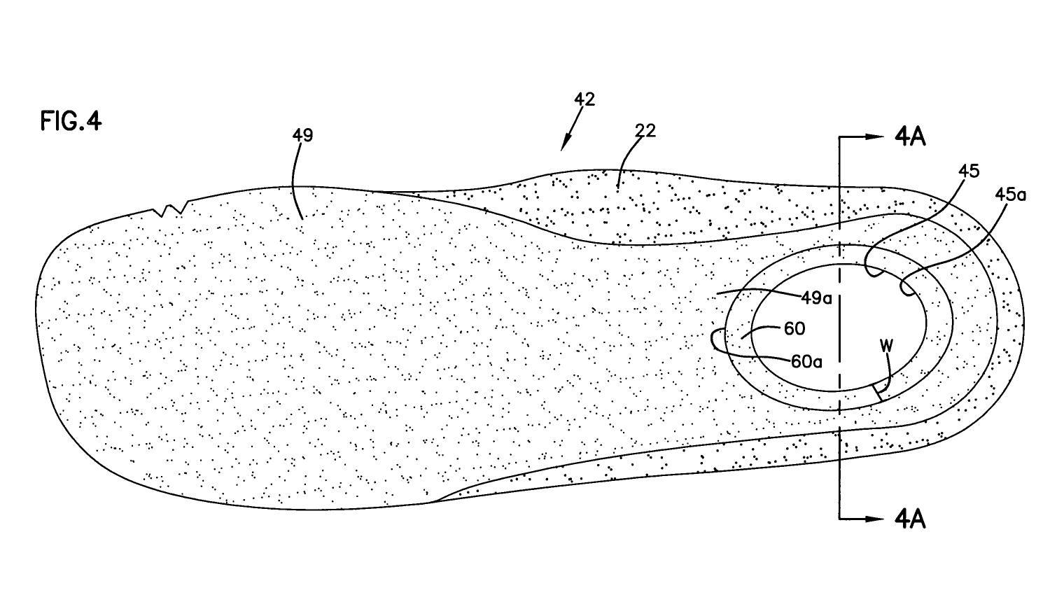

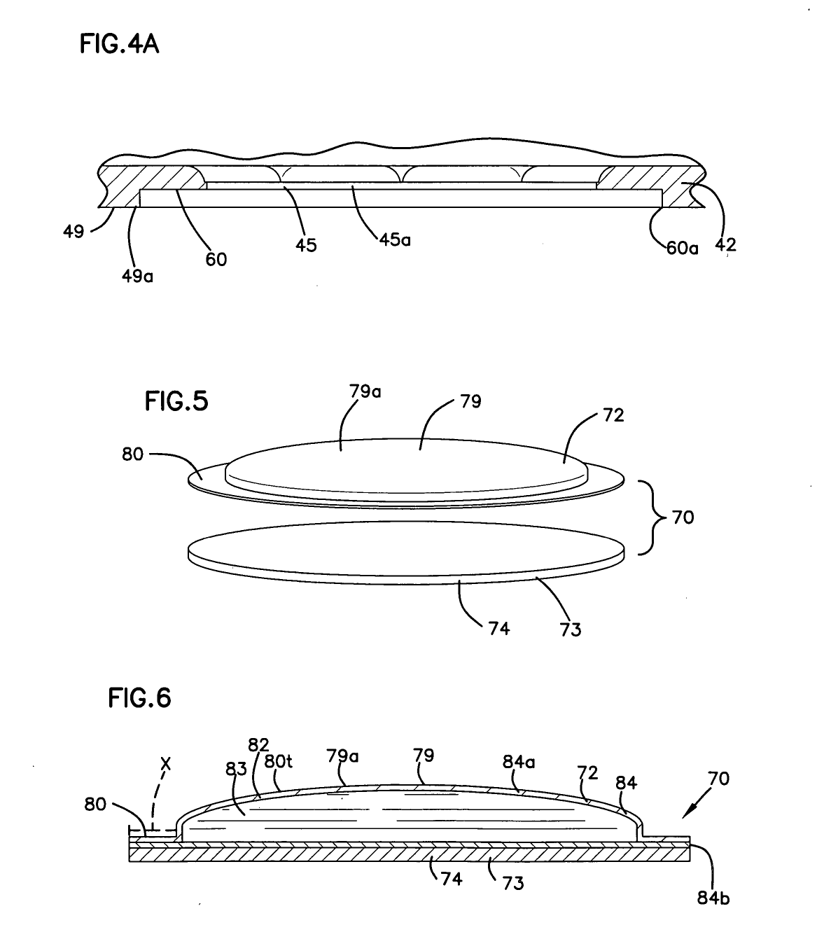

Referring still to FIG. 3, the particular insole base structure 42 depicted, includes a top surface 48 and an opposite bottom surface 49, viewable in FIG. 4. It also includes rim portion 22 surrounding heel periphery support portion 16 and central arch region 19. In FIG. 3, the optional breathe perforations 25 referenced above in connection with descriptions of FIG. 8, are not shown; however they can be formed into (or cut or punched into) the insole base structure 42, FIG. 3, if desired.

Still referring to FIG. 3, the particular insole base structure 42 depicted includes, in heel periphery support portion 16, contoured raised cushion portion(s) 50. The raised portion(s) 50 can comprise cushion material contoured for preferred heel support, and also, if desired, constructed with regions of different softness or density of material therein for example as provided by regions 51 between spaced raised regions 50a. Techniques for accomplishing such structures are described for example in U.S. Pat. No. 6,931,763 B2, filed Aug. 5, 2003, issued Aug. 23, 2005, again incorporated by reference. The raised portions have top surfaces higher than the surface of the remainder of the top surface of the insole base structure. The heel periphery support portion is usually configured to provide stabilizing support, to a peripheral portion of a wearer's heal, around the center cushion 15.

In FIG. 3, the heel periphery support portion 16 has a generally “u” shape, although alternatives are possible. A u-shape will be typical, especially in an arrangement in which the heel cut 45 is oval or elliptical.

A variety of materials and arrangements can be used for the insole base structure 42, including those currently known for slipper insoles. What is, in general, required is an operable insole base structure 42, with an appropriate heel cut 45 therein. However, still referring to FIG. 3, the particular, typical, insole base structure 42 depicted is constructed by laminating a first layer or top layer 54 to a second or bottom layer 55. An insole blank would be formed by such an approach, which can then be used to form the insole base structure 42. Such a footwear insole blank 57 is depicted in FIG. 2, in exploded view so that top layer 54 and bottom layer 55 are separately viewable. The footwear blank 57 can be formed by precutting layers 54 and 55, and then securing them together; or, securing material for the two layers (54, 55) together, and then cutting the footwear blank 57.

Although alternatives are possible, typically the top layer 54 would be a fabric layer 54a, and the bottom layer 55 a foam layer 55b. The two layers 54, 55 can be secured together by a variety of means, for example adhesive. Useable approaches include to apply a dry powder adhesive, hot melt adhesive or a water-based adhesive.

In this context, the words “top” and “bottom”, “upper” and “lower”, “upwardly” and “downwardly” and variants thereof, are generally meant to refer to the relative orientation of components when the footwear member 1, FIG. 8, is positioned on a floor surface with an underside 49 of the sole 10 against the floor. The same is meant when these terms are used in other contexts, herein.

In many typical assembly approaches to formation of the insole base structure 42, the footwear blank 57 is subjected to a compression molding step. Such a process would typically cause a portion of a foam layer 55a to melt, facilitating bonding to a second fabric 54a.

A variety of alternate molding techniques can be used to form an insole base structure 42, however compression molding techniques will be particularly convenient when the insole base structure 42 comprises a fabric layer 54a secured to a foam layer 55a.

The particular material used for the foam layer 55a is a matter of choice, typically based on a selection criteria providing that after the compression molding will provide a desired level of resiliency in the insole of the finished footwear piece 1, FIG. 8.

An example of a usable foam would be an ethylene vinyl acetate foam, such as a sponge ethylene vinyl acetate. The density of the foam layer should be sufficient to provide a desired level of support after the compression molding operation. If the foam density is too low, it is expected that insufficient support to the wearer's foot could result. If the foam density is to high, the insole will feel to rigid and hard to the wearer. Although alternatives are possible, typically the foam material utilized to form foam layer 55a, will be selected from a foam having a density range (before compression molding) between 4 lbs/sq. ft. and 10 lbs/sq. ft. (1.9-4.88 g/sq.cm.)

In a typical application, the foam layer 55a will be provided (before compression molding) as a 2-4 inch (50.8-102 mm) thick block, that will be compressed to form a desired thickness in the insole base structure 42. Foam layer 55a can be provided in pieces if desired. The utilization of pieces allows for the possibility of providing different density of foams, different locations.

The fabric layer 54a can be provided from a variety of fabric materials that will adhere to the foam layer 55a and provide a desired surface structure. The fabric layer can be a woven or nonwoven material. It can be a knitted material. In typical applications, the fabric layer 54a will be visible in the finished product, so it will typically be chosen to a nap (if any) sufficiently small for desired appearance. Typically the nap will be less than about 4 millimeter, although alternatives are possible. An example fabric would be a micro fiber sueded fabric. An example would be polyester fabric.

In some instances sole base structure 42 can be provided without a fabric layer 54a. However, the fabric layer 54a will typically provide an advantageous feel for portions of a wearer's foot, and will also provide durability and desired appearance.

Referring to FIG. 3, in the compression molded insole base structure 42 base portions 50 can be provided by compression molding techniques that can compress regions 51 more than regions 50.

Typically, the rim portion 22 will be formed appropriately, to have the height, i.e., dimension projecting upwardly from an adjacent portion of surface 48 of at least 0.25 inch (6.35 mm) and typically no more than 1.0 inch (25.4 mm).

Typically, the sole base structure 42 will be configured so that, when installed in a footwear shell 2, the front region 20, in particular a region adjacent the toes of a wearer, will be lower than the arch region 19. This provides, among other things, desirable comfort and support. However, alternatives are possible.

Still referring to FIG. 3, aperture 45 is depicted in a center portion of heel periphery support portion 16. Although alternatives are possible, aperture 45 can be formed after the step of compression molding, if desired, by a die cut operation. The particular heel cut 45 depicted, is an oval shaped aperture 45a although alternatives are possible. The particular oval shaped aperture 45a shown, as will be typical, is aligned with a long axis of the oval directed along the length of the insole base structure 42. Although alternatives are possible, this will be typical.

Referring to FIG. 4, bottom side 49 of insole structure 42 is depicted. Aperture 45 is viewable. It is observable from FIGS. 4 and 4a, that aperture 45, on the bottom side 49, is surrounded by a recessed lip structure 60. By “recessed” in this context, it is meant that lip 60 is positioned recessed inwardly from an adjacent portion 49a of surface 49. The amount (depth) of the recess is not critical, but will typically be 1-6 mm, inclusive. The lip would typically have a width dimension W, FIG. 4, of 4-14 mm, inclusive, although alternatives are possible. The recessed lip 60, although not required in all applications described herein, is advantageous for positioning of the heel center cushion 15, as discussed below.

Attention is now directed to FIG. 4A. In FIG. 4A, a cross-sectional view taken along line 4A - 4A, FIG. 4 is shown. In FIG. 4A, the fragmentary cross-sectional view is positioned with underside 49 of insole base structure 42 pointed down, as it would be in normal use by wear. In FIG. 4A, recess shelf 60 can be viewed recessed inwardly with thickness of base structure 42 (i.e., upwardly in the orientation shown).

2. The Heel Center Cushion Portion 15, FIGS. 5-6.

Referring to FIG. 8, footwear piece 1 includes a heel center cushion portion 15 positioned within aperture 45 of insole base structure 42, FIG. 3. As a result, the heel center cushion portion 15 can comprise a different material, than those used to form insole base structure 42. Thus, it is not subject to a requirement, in typical construction, of being a variation of the same materials and same types of materials as used for other portions of the insole base structure 42. Further it need not be subjected to any of the compression conditions, if a compression molding operation used to for insole base structure 42.

A variety of options are available for the heel center cushion portion 15. The heel center cushion portion 15 can comprise a single material, or a composite structure. A variety of materials are usable in the heel center cushion portion including for example: foam cushion materials; gel cushion materials; and, combinations thereof. Typically the heel center cushion portion 15 will include a gel cushion, as discussed below.

An example heel center cushion arrangement 70 usable to form the heel center cushion portion 15 is shown in FIGS. 5 and 6.

Referring first to FIG. 5, the heel center cushion portion includes: upper cushion member 72 and lower cover 73. In some applications, a heel center cushion 15 comprising only a single layer, as opposed to an upper and a lower member, can be used. However, the particular heel center cushion arrangement 70 depicted in FIGS. 5 and 6, is typical for a wide variety of footwear members.

Referring to FIG. 5, lower cover 73, for the example shown, comprises a foam piece 74. The example foam piece 74 has a periphery larger than a periphery 60a of aperture 45 and larger than the inner periphery of the recess lip 60 defined in bottom side 49 of insole base structure 42, FIG. 4. As a result during assembly, lower cover 73 can be positioned to cover aperture 45a and recessed lip 60 in bottom side 49 of insole base structure 42. Alternatives are possible.

Referring to FIG. 4, typically cover 73 would be adhered over recessed lip 60 and aperture 45, by an adhesive or similar means. However, alternate approaches such as melt bonding or melt welding, stitching etc., could be used.

Referring again to FIG. 5, upper cushion member 72 comprises a central cushion 79 in region 79a which will project through heel cut 45 to provide heel center cushion 15, in the footwear piece 1. In the example shown, upper cushion member 72 further includes a peripheral rim 80 sized to engage and overlap recessed lip 60, FIG. 4, during assembly. Thus, rim 80 is preferably sized to fit within a recessed lip 60, for convenient covering by lower cover 73 during assembly. The rim 80 provides for a mechanical interlock, preventing the cushion arrangement 70 from pressing completely through aperture 45, during assembly or use. A typical rim 80 would have a width, dimension X, FIG. 6, of 3-14 mm, although other alternatives are possible.

A variety of constructions can be used for upper cushion member 72, including single materials or composite constructions. Foam materials, for example, can be used. It is anticipated that in typical applications, for the current slipper market, the upper cushion member 72 will comprise a gel pack, typically comprising a polymeric cushion material secured within an outer envelope, package or covering.

Herein the term “gel pack” is meant to refer to a cushion package that includes a polymeric cushion having gel-like physical properties for cushioning. Typically these properties include an ability to compress appropriately under the heel of wear, and, to rebound to an original shape or nearly to an original shape, after the pressure is relieved. Some examples are described below.

Attention is directed to FIG. 6, in which a cross-sectional view of heel center cushion arrangement 70 is depicted. Here upper cushion member 72, in the example shown, is depicted as a gel pack 82 including a polymeric cushion 83 secured within packet 84. The packet 85 would typically comprise an open central volume with an outer rim sealed by heat sealing or similar techniques. An example would be to use vinyl layers 84a, 84b to form the packet 84, with the polymeric cushion 83 contained therein.

The gel or polymeric cushion 83 could comprise a variety of materials appropriate for utilization as footwear supports. An example would be a polyurethane material, although alternates are possible.

Usable materials are discussed below in Section III.

Typically, the upper peripheral area (upper perimeter) of central cushion 79, FIG. 6, i.e., the upper perimeter defined by cushion 79, is within the same ranges of areas discussed above, for central aperture 45a, i.e., at least 1,100 sq. mm., typically not more than 1,800 sq. mm., often within the range of 1,200-1,600 sq. mm., for example 1,250-1,500 sq. mm., inclusive. Of course alternatives for both the aperture and the heel cushion perimeter are possible.

The exposed upper surface of the heel center cushion arrangement may be positioned above the upper surface of the raised regions within the heel periphery support portion. For example, the exposed upper surface of the heel center cushion arrangement may be 1 to 3 mm higher than the upper surface of the raised regions. Alternatively, the raised regions and the exposed upper surface of the heel center cushion arrangement may be at the same height. In another alternative, the upper surface of the raised regions is higher than the exposed upper surface of the heel center cushion arrangement.

3. Assembly of the Completed Footwear Piece 1.

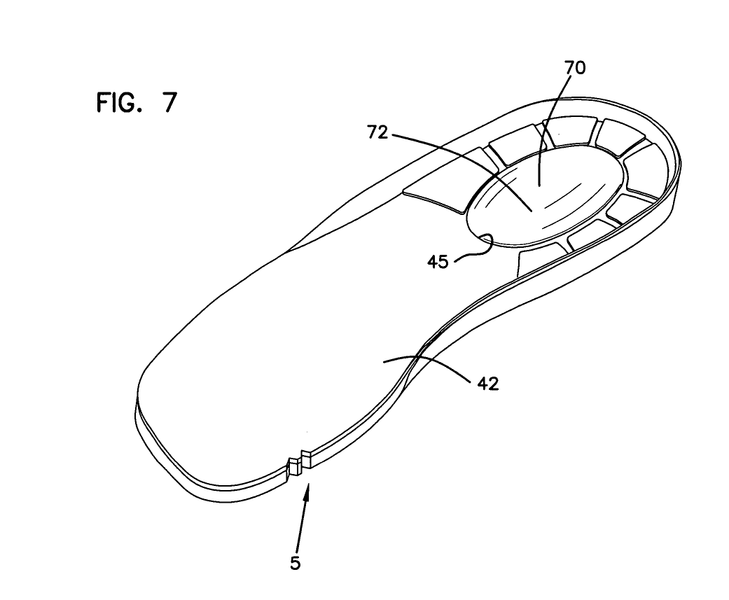

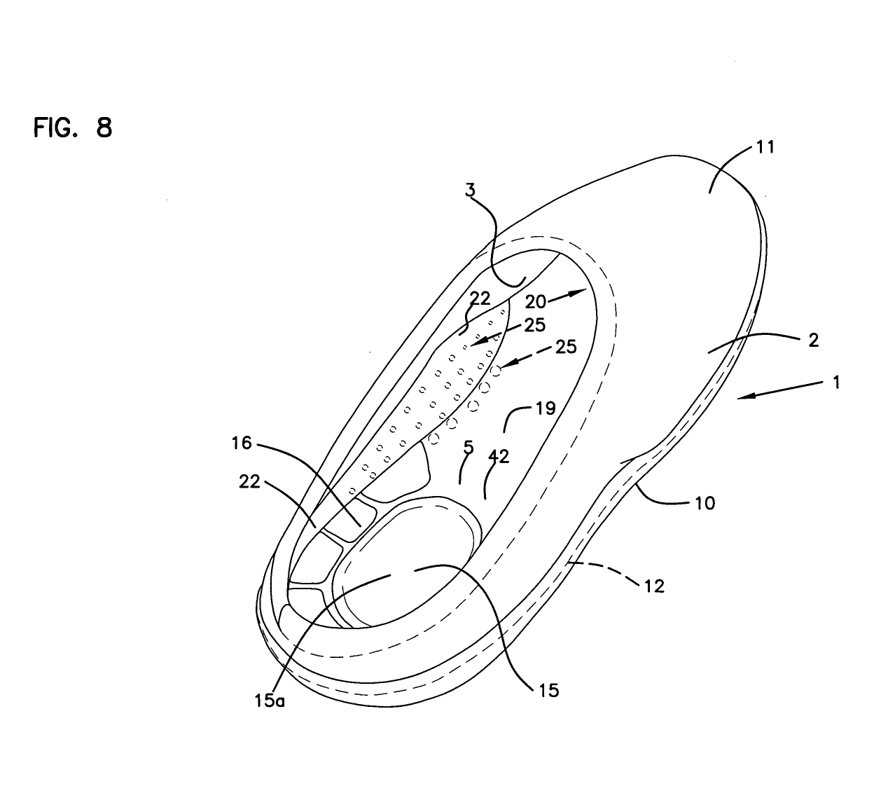

Assembly of the completed footwear piece 1, will be apparent from the previous descriptions. The heel center cushion arrangement 70 is positioned in engagement with the insole base structure 42, typically by inserting the upper cushion member 72 to project upwardly through aperture 45, and then covering upper cushion member 72 with lower cover 73, securing lower cover 73 in place for example by adhesive. In FIG. 7, a footwear sole insert structure arrangement 5 resulting from such an assembly is depicted.

The footwear sole insert structure arrangement 5 could then be inserted within shell 2, typically being secured in place by an adhesive or stitching. The result is footwear piece 1, FIG. 8.

### III. Sample Materials and Dimensions

A. Example polymeric cushion materials

As indicated above, a variety of materials can be used in the heel center cushion arrangement, to provide cushioning to the heel. It is expected that polymeric cushion having gel-like physical properties will be typically be utilized for arrangements according to the present disclosure.

An example, as indicated above, would be to position a polymeric cushion 83 within a packet formed from a vinyl material. Typically at least an upper layer 80t (FIG. 6) for the gel pack 70, would be made from a clear vinyl material, to allow inspection of the polymeric cushion 83.

The polymeric cushion typically is an elastomer which has a hardness value measured on the Shore 00 scale of less than 75, typically less than 35, and usually less than 25. The polymeric cushion 83 typically has a Shore 00 value which is greater than zero.

The hardness of elastomeric materials are frequently tested using a Shore A Scale Durometer, as described in ASTM D 2240. The Shore Durometer uses a short indenter (0.098 inches in length) that is pressed onto a sample by an 822 gram spring. Each 0.001 inch of deflection is shown as 1 degree Shore A. As the material tested becomes harder, the amount of deflection (and therefore the Shore value) increases. The Shore 0 scale is used for testing materials below 10 degrees while the Shore 00 scale is used for testing materials registering 10 or less on the Shore 0 scale. The Shore 00 scale requires use of a spherical indenter and a 225 gram spring. A suitable sample for test is about 6 millimeters thick and has a surface area sufficient to permit at least three test points that are at least 5 millimeters apart and at least about 13 millimeters from any edge.

The polymeric cushion 83 is typically a soft polyurethane polymer. Polymers having Shore 00 values below about 75 are easily deformed yet rapidly recover their initial shape once released.

Preferably, the polymeric cushion 83 has an elongation to break value of at least about 500 percent. This is an indication of the elasticity of the polymer. The polymers used in the cushion 83 can preferably be stretched to at least 5 times their original size. Preferably, the polymeric cushion 83 used has a compression set of less than about 15 percent. A compression set of less than about 15 percent means that the polymer will regain at least about 85 percent of its original shape or size once the deforming force is removed.

Polyurethane polymers are formed from the reaction of isocyanate containing components and active hydrogen-containing components. Typically, the hydrogen-containing components are polyols. Certain polyurethanes which can be used for cushion 83 can be produced by mixing polyols and polyisocyanates at ambient temperature in a mixing head. At the exit of the mixing head, the chemical mixture is typically a liquid with a viscosity between about 1 and 1000 poise. The polyols and isocyanates react together in an exothermic reaction to form a cross-linked network of polyurethane polymer. No heat needs to be added; so manufacturing costs can be kept low.

A typical density for a useable polyurethane materials is 1.07 g/cu.cm., as measured by ASTM standard ASTM-D792, although alternatives are possible. Typically the density would be within the range 1.0-1.12 g/cc.

The maximum thickness of the gel packet 83 is a matter of choice. In typical applications it will have a thickness within the range of 0.30-0.4 inch (7.6-10.2 mm), although alternatives are possible. The longest dimension of the upper surface 79 of the gel pack 70 would typically be within the range of 1.75 inch -3.5 inch (44-89 mm), and the shorter dimension 0.75-1.5 inch (19-38.1 mm) although alternatives for each are possible.

Examples of suitable polymers and the synthesis thereof are described in U.S. Pat. Nos. 4,346,205, 4,722,946, and 5,362,834, the disclosures of these patents being hereby incorporated by reference. Commercially available polymers are available from such companies as Pittsburgh Plastics, Sorbothane, Viscolas, and Bayer. A low hardness grade polymer useful as gel 83 is available under the name ISOGEL®.

B. The bottom sole 10.

The bottom sole 10 is typically a low-density polyolefine or styrene block copolymer, such as exemplified by the Kraton® materials produced by Shell. Adherence between the outsole and the polymer insole jell simplifies manufacture.

### IV. Additional Comments and Observations

In general terms, a footwear member is provided, typically in the form of a slipper. The footwear member includes a footwear shell and a footwear insole insert structure arrangement. The footwear shell typically comprises a sole and an upper combined to define a footwear shell interior. The upper can provide for an open toe or a closed toe construction.

The footwear insole structure arrangement includes an insole base structure and a heel center cushion arrangement. The base structure include a heel cut, and the heel center cushion arrangement is positioned within the heel cut to project upwardly therefrom. As a result the heel center cushion arrangement provides or defines an exposed upper surface, for contact with the heel of a wearer.

The typical heel center cushion arrangement would include a gel pack positioned to define the exposed upper surface. The typical heel cut would be an aperture in the insole base structure.

An example is shown in the figures.

In the context of the previous descriptions and examples shown in the figures, the following characteristics and observations can be obtained, for a typical example having all of the types of features shown:

1. Assembly utilizing a gel pack appropriately positioned is convenient, since the heel cut aides in securely positioning the gel pack, even though the gel pack is substantially smaller than the insole base structure and outer periphery. Similarly, integrity is maintained after assembly.

2. Manufacture of the insole base structure is conducted independently of the heel cushion arrangement (for example gel pack), which is convenient, since the heel cushion arrangement is not subjected to the conditions of compression molding, for the insole base structure.

3. Inspection to assure proper positioning and integrity to the gel pack is convenient both in the manufacturing process and for the consumer(s).

4. When implemented with a heel periphery support section, comfort in which a center of the heel is cushioned differently from an outside of the heel is possible.

5. There is no need to form, mold or cut a recess in the sole 10 (or materials positioned above the sole 10) to securely retain or receive the heel cushion arrangement.

It is noted that a footwear arrangement does not need to include all of the features characterized herein, to obtain some improvement or advantage according to the present disclosure.

What is claimed is:

1. A footwear member comprising:
(a) a footwear shell comprising a sole and an upper defining a footwear shell interior;
(b) a footwear insole insert structure arrangement secured within the footwear shell interior; the footwear sole insert structure arrangement comprising an insole base structure and a heel center cushion arrangement;
(i) the insole base structure including a heel cut therethrough; and
(ii) the heel center cushion arrangement being positioned within the heel cut define an exposed upper surface.

2. A footwear member according to claim 1 wherein:
(a) the heel cut is an aperture through the insole base structure and surrounded by a portion of the insole structure.

3. A footwear member according to claim 1 wherein:
(a) the insole base structure includes:
(i) a heel periphery support portion;
(ii) a central arch region;
(iii) a front section; and,
(iv) a rim portion adjacent the central arch region and the heel periphery support portion.

4. A footwear member according to claim 3 wherein:
(a) the heel periphery support portion is contoured with spaced, raised, portions.

5. A footwear member according to claims 1 wherein:
(a) the insole base structure comprises a compression molded laminate of fabric and foam.

6. A footwear member according to claim 1 wherein:
(a) the heel center cushion arrangement comprises a gel pack including a polymeric cushion material.

7. A footwear member according to claim 1 wherein:
(a) the heel center cushion arrangement comprises a gel pack positioned over a foam piece.

8. A footwear member according to claim 7 wherein:
(a) the heel center cushion arrangement has an upper perimeter area of at least 1100 sq. mm. and not more than 1800 sq. mm.

9. A footwear member according to claim 1 wherein:
(a) the heel center cushion arrangement has an upper perimeter area within the range of 1200 to 1600 sq. mm., inclusive.

10. A footwear member according to claim 1 wherein:
(a) the heel center cushion arrangement has an oval shaped upper perimeter.

11. A footwear member according to claim 1 wherein:
(a) the heel center cushion arrangement includes a gel pack comprising:
(i) an outer flexible package defining an interior; and
(ii) a polymeric cushion positioned within the interior of the outer flexible package.

12. A footwear member according to claim 11 wherein:
(a) the polymeric cushion comprises a polyurethane polymer.

13. A footwear member according to claim 12 wherein:
(a) the polymeric cushion has a hardness Shore 00 scale, of no greater than 25.

14. A footwear member according to claim 11 wherein:
(a) the outer flexible package comprises a vinyl package.

15. A footwear member according to claim 11 wherein:
(a) the outer flexible package of the gel pack includes a perimeter rim projection having a width of 2-10 mm.

16. A footwear member according to claim 1 wherein:
(a) the insole base structure includes an upper surface and a lower surface;
(b) the heel cut is an aperture projecting through the insole base structure;
(c) on a lower surface, the insole base structure includes a recessed shelf region surrounding the heel cut; and,
(d) the heel center cushion arrangement comprises a gel pack having a peripheral rim;
(i) the gel pack being positioned with the peripheral rim adjacent to, and underneath, the recessed shelf region and with a central portion of the gel pack projecting upwardly through the aperture.

17. A footwear member according to claim 16 wherein:
(a) the heel center cushion arrangement includes a foam bottom cover positioned underneath the insole base structure.

18. A footwear member according to claim 17 wherein:
(a) the footwear shell includes a sole cushion positioned above the sole and underneath the footwear insole insert structure.

19. A footwear member according to claim 18 wherein:
(a) the insole base structure includes a plurality of breathe apertures positioned in at least one of: a central arch region; a front region; and, a rim section adjacent the central arch region.

20. A footwear member comprising:
(a) a footwear shell comprising a sole and an upper defining a footwear shell interior; and
(b) a footwear sole insert structure arrangement secured within the footwear shell interior; the footwear sole insert structure arrangement comprising an insole base structure and a heel center cushion arrangement;
(i) the insole base structure including a heel portion with a central heel aperture projecting therethrough;
(A) the central heel aperture having an area within the range of 1100 to 1800 sq. mm, inclusive.
(B) the heel portion including a contoured heel periphery support portion having a contoured upper surface with first, thick, regions and second, thin, regions; and,
(ii) the heel center cushion arrangement including a gel pack positioned surrounded by the central heel aperture and projecting upwardly from the thin regions of the insole base structure.
